(19) United States
(12) Patent Application Publication
(10) Pub. No.: US 2003/0215513 A1
(43) Pub. Date: Nov. 20, 2003

(54) Method for releasing nanosized particles of an active substance from a diffusion-controlled pharmaceutical composition for oral use

(76) Inventors: Peter Fyhr, Brosarp (SE); John Kendrup, Oxie (SE); Johan Borgstrom, Oxie (SE); Maria Nilsson, Malmo (SE)

Correspondence Address: EDWARDS & ANGELL, LLP, P.O. BOX 9169, BOSTON, MA, 02209, US

(21) Appl. No.: 10/372,045

(22) Filed: Feb. 20, 2003

(30) Foreign Application Priority Data
Feb. 21, 2002 (DK) PA 2002 00275

(51) Int. Cl. A61K 31/7024; A61K 31/56; A61K 31/55; A61K 31/455; A61K 31/426; A61K 31/337; A61K 31/522

(52) U.S. Cl. 424/489; 514/355; 514/179; 514/217; 514/53; 514/369; 514/449; 514/263.38

(57) ABSTRACT

The present invention relates to a method for releasing an active substance from a composition. The active substance is either substantially water-insoluble and/or immobilised on or in nanosized particles. A diffusion gradient is established between the inside and the outside of the composition, which allows the transport of a nanosuspension of the active substance through the pores of the membrane.

### FIELD OF THE INVENTION

The present invention relates to a method for releasing an active substance, in particular in nanosized form, from a pharmaceutical composition that is coated with a diffusion-controlled membrane that contains a multiplicity of pores or a pore-forming substance. The invention is especially suitable for active substances that are either substantially water-insoluble or active substances that have been immobilized on substantially water-insoluble nanosized particles.

### BACKGROUND OF THE INVENTION

It is well known that many drug substances of today have a very poor solubility in aqueous media. Such drug substances normally have a variable therapeutic effect due to a variable and incomplete bioavailability after e.g. oral administration. It is generally contemplated that a prerequisite for obtaining a therapeutic effect of a drug substance is that the drug substance at least partly is present in the body in dissolved form. In order to reduce such problems many attempts have been made in order to increase the solubility and/or dissolution rate of the drug substance in aqueous media, e.g., by salt, complex or prodrug formation or by providing the active substance in micronized form.

The present invention addresses this problem and provides a solution by which the drug substance is released as nanosized particles from a solid oral dosage form.

### DISCLOSURE OF THE INVENTION

The present invention relates to a method for releasing an active substance, which is substantially water-insoluble and/or immobilised on or in nanosized particles, from a pharmaceutical composition that is coated with a diffusion-controlled membrane that contains a multiplicity of pores or a pore-forming substance, the method comprising

i) contacting the pharmaceutical composition with an aqueous solvent (e.g. gastro intestinal fluids),

ii) diffusion of the solvent into the pharmaceutical composition so that a) one or more water-soluble substances contained in the pharmaceutical composition is at least partly dissolved to obtain one or more solutes, and b) one or more substantially water-insoluble nanosized active substances or aggregates thereof, or water-insoluble nanosized particles containing the active substance is at least partly suspended in an aqueous medium, to obtain a nanosuspension of nanosized particles,

iii) diffusion of the one or more solutes through the diffusion-controlled membrane and out of the pharmaceutical composition,

iv) establishing a diffusion gradient that enables a mass transport of the nanosuspension from the pharmaceutical composition through pores in the diffusion-controlled membrane,

whereby the active substance is released from the composition.

The pharmaceutical composition is generally in the form of a solid dosage form for oral intake such as, e.g. in the form of tablets, capsules, pellets, sachets or the like. It may be in the form of a single unit dosage form (e.g. a matrix tablet) or in a multiple unit dosage form (e.g. in the form of pellets or sachets or tablets or capsules comprising pellets). The pharmaceutical composition is provided win a diffusion-controlled coating containing a multiplicity of pores or containing a pore-forming substance that creates a multiplicity of pores upon contact with an aqueous medium. To the best of our knowledge it has not previously been possible to deliver active substances in particular form through a diffusion-controlled membrane. Normally, only diffusion of dissolved material takes place through a diffusion-controlled membrane and in such cases the limiting factors in releasing substantially water-insoluble active substances are normally the dissolution rate and/or the solubility of the active substance in the aqueous medium that has diffused into the pharmaceutical composition. Other kinds of release systems have been developed such as, e.g., release systems based on osmotic pressure. However, the idea behind the method of the present invention differs from such known systems e.g. in the presence of a multiplicity of pores in the membrane and in that the release from the composition takes place by diffusion through the membrane.

Compared to known drug delivery systems for oral use, a pharmaceutical composition based on the present release mechanism has several advantages.

Firstly, such pharmaceutical compositions can be employed to control the dissolution rate and, accordingly, the release rate of poorly or moderately soluble drug substances. Upon release of the nanosized particles from the composition, the exposure of the active substance to an aqueous environment is increased (as compared to the relatively small amount of aqueous medium present within the composition) leading to improved conditions with respect to dissolution of the active substance. The membrane porosity, and mainly the fraction of pores spanning through the membrane, can be varied to control the transport of the nanosized particles through the membrane. The pharmaceutical composition thus allows better possibilities for controlling the release rate of the active substance than for example a pharmaceutical composition in the form of a matrix composition comprising the nanosized particles. Furthermore, the composition is not subject to erosion in contrast to e.g. matrix-based systems.

Secondly, the interior of a pharmaceutical composition based on the present release mechanism (i.e. the material inside the diffusion-controlled membrane and enclosed by the membrane) contains normally a mixture of pharmaceutically acceptable excipients and one or more active substances. Careful selection of these ingredients allows a highly controlled environment from which the active substance in nanosized particular form is released. For example inclusion of a buffer substance allows control of pH in the interior of the pharmaceutical composition (irrespective of the pH of the aqueous medium diffusing into the interior of the composition). Furthermore, the conditions inside the composition can be selected to control the formation and stability of the nanosuspension. Moreover, as it will appear from the discussion under the heading “Description of the release of nanosized particles” many other means for controlling the release of active substance from the composition are possible.

Thirdly, the membrane and the interior conditions essentially control the release rate of the nanoparticles. Thus, a change in the pH outside the composition (e.g. influenced by the intake of food or by the different parts of the gastrointestinal tract through which the composition passes) may have only little or no effect on the release rate.

Fourthly, the manufacturing costs are relatively low and the techniques employed are well known in the art of pharmaceutical formulation. Moreover, it is possible to obtain a large flexibility in release rate, i.e. a composition can be designed for fast release or for slow release etc. The release mechanism applies for e.g. tablets as well as pellets, i.e. the technology is also flexible.

The method according to the present invention allows the release of the active substance from the pharmaceutical composition to be controlled. For example, zero or first-order release of the active substance from the pharmaceutical composition may be obtained. Alternatively, the active substance may be released immediately.

In the following the term “Nano-DCV” is denoted for the above-mentioned technology, i.e. delivery of active substance in the form of nanosized particles from a diffusion-controlled composition such as diffusion-controlled vesicles (DCV).

Active substances

As mentioned above, a pharmaceutical composition based on the present release mechanism contains an active substance. In the present context the term “active substance”0 is intended to include therapeutically, prophylactically and/or diagnostically active substances including any biologically and/or physiologically active substance that has a function on an animal such as, e.g., a mammal like a human. The term includes drug substances, hormones, genes or gene sequences, antigen-comprising material, proteins, peptides, nutrients like e.g. vitamins, minerals, lipids and carbohydrates and mixtures thereof. In other words, the term includes substances that are useful in the treatment, prophylaxis and/or diagnosis of diseases or disorders affecting animals or humans, or in the regulation of any physiological condition. The term also includes any biologically active substance that has an effect on living cells or organisms.

An active substance for use in a method of the present invention is normally substantially water-insoluble, but as has been discussed hereinbefore, the active substance may also be water-soluble. In the latter case, the active substance is normally immobilised on water-soluble polymer nanosized particles. In general, the substantially water-insoluble active substance or the substantially water-insoluble nanosized particles carrying the active substance has a water-solubility of at the most about 20 mg/ml in water at 37° C. such as, e.g., at the most about 15 mg/ml, about 10 mg/ml, about 7.5 mg/ml, at the most about 5 mg/ml, at the most about 3 mg/ml, at the most about 1 mg/ml, at the most about 0.5 mg/ml, at the most about 0.25 mg/ml, at the most about 0.1 mg/ml or at the most about 0.05 mg/ml.

The therapeutically, prophylactically and/or a diagnostically active substance may also be in the form of a pharmaceutically acceptable salt, solvate or complex thereof or in any suitable crystalline or amorphous form or it may be in the form of a prodrug.

Examples of active substances suitable for use in the present context include e.g. antibacterial substances, antihistamines and decongestants, anti-inflammatory agents, antiparasitics, antivirals, local anesthetics, antifungals, amoebicidals or trichomnonocidal agents, analgesics, antianxiety agents, anticlotting agents, antiarthritics, antiasthmatics, anticoagulants, anticonvulsants, antidepressants, antidiabetics, antiglaucoma agents, antimalarials, antimicrobials, antneoplastics, antiobesity agents, antipsychotics, antihypertensives, antitussives, auto-immune disorder agents, anti-impotence agents, anti-Parkinsonism agents, anti-Alzheimers' agents, antipyretics, anticholinergics, anti-ulcer agents, anorexics, beta-blockers, beta-2 agonists, beta agonists, blood glucose-lowering agents, bronchodilators, agents with effect on the central nervous system, cardiovascular agents, cognitive enhancers, contraceptives, cholesterol-reducing agents, cytostatics, diuretics, germicidals, H-2 blockers, hormonal agents, hypnotic agents, inotropics, muscle relaxants, muscle contractants, physic energizers, sedatives, sympathomimetics, vasodilators. vasoconstrictors, tranquilizers, electrolyte supplements, vitamins, counterirritants, stimulants, anti-hormones, drug antagonists, lipid-regulating agents, uricosurics, cardiac glycosides, expectorants, purgatives, contrast materials, radiopharmaceuticals, imaging agents, peptides, enzymes, growth factors, etc.

Specific examples include active substances selected from the group consisting of nifedipine, felodipine, amiodipine, nisoldipine, isradipine, amiodipine, nicardipine; most steroid hormones such as, e.g., estrogen, progesterone, testosterone and derivatives and analogues thereof such as desogestrel, mesterolon, ebnylestradiol, ethinyestradiol, nandronion, nandronlone, hormone antagonists such as tamoxifene, toremifene, flutamide, nilutamide; glucocorticoids such as, e.g., cortisone, hydrocortisone, fludrocortisone, fludocortisone, betametasone, prednisolone, budesonide, and neurological drugs such as carbamazepine, carisoprodol, primidone, zonisamide, perphanazin, antidiabetic drugs such as gilbencdamide, gilmepiride, gliplzlde, and miscellaneous poorly-soluble drugs such as sucralfate, paclitaxel, and acyclovir and all orally absorbable drugs with a water solubility less than about 10 or 20 mg/ml.

As mentioned above, the particle size of the active substance or of the nanosized particles employed carrying the active substance is important it must be of a relatively small size in order to pass the diffusion-controlled membrane and release the active substance outside the composition. The release of the active substance takes place in the gastrointestinal tract after oral administration and then the active substance can e.g. enter into the systemic circulation in order to exert a therapeutic, prophylactic or diagnostic response. The active substance or the nanosized particles employed in the composition has a volume weighted median particle size of at most about 2000 nm such as, e.g., at the most about 1500 nm, at the most about 1000 nm, such as, e.g., from about 1 nm to about 1000 nm, from about 2 nm to about 750 nm, from about 5 nm to about 500 nm or from about 7.5 nm to about 500 nm, from about 10 nm to about 500 nm, from about 50 nm to about 500 nm, from about 75 nm to about 400 nm, or from about 100 nm to about 300 nm as measured by static light scattering/diffraction or dynamic light scattering.

The amount of active substance incorporated in a pharmaceutical composition may be selected based on principles well known in the art of pharmaceutical formulation. The amount depends inter alia on the individual active substance, on the therapeutically effective dosage, the age and condition of the patient and on the disease or condition to be treated. The amount also depends on whether the pharmaceutical composition is designed for administration once, twice, three times or more daily or with a less frequent dosage administration regime.

Diffusion-controlled coatings

The diffusion-controlled membrane of a pharmaceutical composition based on the present release mechanism comprises a substantially water-insoluble polymer. The polymer is selected from the group consisting of i) cellulose derivatives including cellulose esters such as, e.g. ethylcellulose, cellulose acetate, cellulose acetate butyrate, cellulose acetate propionate, cellulose propionate, cellulose butyrate, cellulose valerate, nitrocellulose, ii) acrylic polymers such as, e.g., polymethyl methacrylate, poly(ethylacrylate, methylmethacrylate, trimethylammonloethylmethacrylate chloride), poly(ethylacrylate, methylmethacrylate), iii) vinyl polymers such as, e.g. polyvinyl polymers such as, e.g polyvinyl acetate, polyvinyl formal, polyvinyl butyryl, vinyl chloride-vinyl acetate copolymer, ethylene-vinyl acetate copolymer, vinyl-chloride-propylene-vinyl acetate copolymer, polyvinyl chloride, polyvinyl chloride terpolymers, iv) other polymers such as, e.g. polyethylenes, polypropylenes, polyisobutylenes, polycarbonates, polybutadienes, polyesters and other high molecular synthetic polymers and block-, copolymers and combinations thereof.

The concentration of the polymer in the finished coating is generally from about 10% to about 70% wtw such as, e.g. from about 10% to about 65% w/w or from about 15 to about 50% w/w.

Virtually any coating (membrane) that has or can form pores with a pore size larger than about 100 nm in diameter or larger can be used for the claimed invention. As examples of such membranes, three different membranes are described in the examples herein.

The coating may contain various excipients such as, e.g., plasticizers such as e.g. acetyltributyicitrate, tributylcitrate, triacetin, acetyltriethylcitrate, triethylcitrate, oleic acid, dibutyl sebacetate, diethyl phthalate, benzyl benzoate, polyethylene glycol, triglycerides such as, e.g., hydrogenated vegetable oils, raffinated vegetable oils or glyceryl tracetate, anti-adhesives such as, e.g., silicium dioxide, inert fillers, lipophilic agents such as, e.g. stearic acid, capric acid or hydrogenated castor oil, pigments etc.

The plasticizer is normally incorporated in the coating in a suitable concentration to reach a desired glass transition temperature (Tg) and minimum film formation temperature (MFT). Normally, the desired temperatures are about 10-30° C. and about 0-40% w/w of plasticizer is required.

In some cases, the coating may form a multiplicity of pores without any further means, However, in general it is necessary to include a pore-forming substance in the coating. In the present context a pore-forming substance is a substance that has a suitable water-solubility, i.e. it will dissolve when the pharmaceutical composition is brought into contact with an aqueous medium and as a result a multiplicity of pores will be formed in the coating. It is envisaged that the pores generally are homogeneously distributed in the coating that encloses the core composition. The size of the pores depends on the size of the pore-forming substance and of the distribution of the pore-forming substance in the coating. Thus, the pores are not expected to be of the same size and the pores may also be so formed that a more or less regular channel is formed from the outside of the coating to the interior. Accordingly, the coating dispersion comprises a dispersion of a substantially water-insoluble polymer and a water-soluble pore-forming substance and, optionally other additives like e.g. a plasticizer. The pore-forming substance is normally suspended in the coating composition although there may be situations where the pore-forming substance is dissolved in the coating composition.

The diffusion-controlled membrane may be applied on the composition in the form of a coating dispersion. Normally, in the case of a water-based coating composition with suspended pore former, the coating comprises a pore-forming substance that in the coating composition, which is applied on the pharmaceutical composition, has a solubility of at the most about 100 mg/ml such as, e.g., at the most about 50 mg/ml or at the most about 10 mg/ml at room temperature. In the case of an organic solvent based coating composition, there are no limitations with respect to water-solubility. In the case of a water-based coating composition with dissolved pore former, the solubility must be sufficiently high in order for the pore former to dissolve in the coating dispersion.

The pore former can be dissolved or suspended in the coating liquid. Generally, suspended pore-forming substance has a mean particle size of from about 0.1 to about 500 μm such as, e.g. from about 0.5 to about 100 μm or from about 1 to about 25 μm. The concentration of the polymer in the coating is generally from about 10-70% w/w such as, e.g. from about 10 to about 65% w/w or from about 15 to about 50% w/w.

The pore-forming substance is normally present in the finished coating in a concentration corresponding to from about 0 to about 90% w/w of the total weight of the coating (dry matter).

The pore former can be any substance that has a sufficient solubility to be dissolved by the gastrointestinal fluid. Examples are sucrose and other sugars, urea, salts such as potassium chloride, sodium chloride, calcium chloride, sodium phosphates (basic, dibasic and monobasic), potasium phosphates (basic. dibasic and monobasic), calcium sulphate, sodium sulphate, sodium citrates (basic, dibasic and monobasic), sodium tartrates (monobasic and dibasic), potassium tartrates (monobasic and dibasic), soluble polymers such as polyvinyl pyrrolidone, methyl cellulose, hydroxy propyl methyl cellulose, hydroxy propyl cellulose, hydroxy ethyl cellulose, polyvinyl alcohol, chitosan, poly(butylmethacrylate), (2-dlmethyl aminoethyl)-methacrylate, methyl methacrylate dextran, maltodextrin, xanthan, potassium salts, calcium salts, magnesium salts, amino acids, weak adds, carbohydrates, polymers with amino and/or acid functions and combinations thereof. Other examples include potassium bitartrate, potassium hydrogen tartate, creatine, asparagine, glutamine, aspartic acid, glutamic acid, leucin, neroleucine, norleucine, inosine, isoleucine, magnesium citrate, magnesium phosphate, magnesium carbonate, magnesium hydroxide, magnesium oxide, magnesium salts and the like and combinations thereof. The pore-forming substance according to the present invention has a solubility in the coating dispersion of at the most about 100 mg/ml such as, e.g., at the most about 50 mg/ml or at the most about 10 mg/ml at room temperature.

The coating (i.e. the membrane) may be applied on the composition in the form of a coating composition. The pore-forming substance may be suspended and to the major part remains un-dissolved in the coating dispersion. The coating composition normally contains a solvent such as, e.g., an organic solvent like e.g. acetone, ethanol, isopropanol and the like, or an aqueous solvent like e.g. water. The choice of polymer and/or pore-forming substance necessitates the use of either an aqueous or an organic solvent for the coating composition. In principle, all polymers can be used irrespective of the solvent employed. In practice, however, the polymer employed in aqueous media may be in the form of a latex Especially, pore-formers like potassium hydrogen tartrate, creatine etc. are suitable for use in systems with suspended pore former in a water-based coating suspension.

With respect to the thickness of the coating (membrane), the cores normally gain about 10% w/w when coated (normal range from about 1 to about 30% w/w such as, e.g., from about 5% to about 20% w/w).

Description of coating with aqueous or solvent based membranes

The process of applying a membrane on a pharmaceutical preparation (here denoted as core) is known as coating. In the present invention there is a great variety regarding which cores that can be used. Depending on the desired result multiple units (for example crystals, beads, granules, pellets, mini tablets, sachets) and single units (pills, tablets, capsules) may be used. The coating is commonly executed by spraying a liquid, containing the components of the finished coating, onto the cores. During the process the main part of the liquid dries off. The two most common techniques for coating are the fluid-bed type (e.g. tangential-spray, top-spray, bottom-spray, Wurster process, rotating bottom, Kugelcoater) and the rotating pan type (e.g. Accela cota, sugar coating). Small cores are predominately coated in the fluid-bed type and large cores predominately in the rotating pan type.

Prior to the coating process the coating dispersion must be prepared. It is important to properly disperse the ingredients. Depending on the ingredients that are used, different approaches are needed for the dispersion. Agitation is not needed for all coating dispersions but most of the dispersions need either cautious or vigorous stirring. Cautious stirring can for example be achieved with magnetic stirring or with a propeller. The latter are often used together with baffles to minimize foam formation. Vigorous stirring can for example be achieved with Ultra-Turrax, Turbine mixers and homogenisers Vigorous stirring can be needed to speed up dissolution of some ingredients and to properly disperse (deagglomerate) other ingredients such as pigments and some pore formers.

The coating liquid can be either predominately water based or organic solvent based. Water based coatings, e.g. latexes, are often quite sensitive towards variations in process parameters. The temperature of the cores should be above minimum film formation temperature but sufficiently low to minimize sicking. Above the glass transition temperature of the polymer, the polymer gets sticky. Therefore, the temperature often is from about 20° C. to about 40° C., but depending on the coating, the proper core temperature may be below or exceed these temperatures with several degrees.

Prior to the coating, the cores are placed in the coating equipment The cores can be pre-heated or not. During the coating process the coating dispersion may be stirred. If the dispersion contains suspended pore former or other suspended material, stirring is preferably used. When the suitable amount of coating dispersion has been applied, the coated cores can be further treated in different ways. It is common to dry the coated cores in the coating equipment and/or in another drying equipment, in order to dry off more liquid. Further treatment can also be used to cure the coated cores. The type of curing can be chosen to obtain desired stability, release-rate, coating strength and/or other properties.

The pores in the surface of the cellulose acetate membrane can be distinguished as large dark areas in FIG. 1, whereas the cross-sectional images of the solvent based membranes show the dark pores much more clearly, see FIG. 2 a and b. Clearly, the structure and porosity of the membranes varies significantly between different types of membranes.

A difference in the amount as well as the size distribution of the pores was found between the solvent-based membranes with high and low porosity. This indicates that the porosity, i.e. concentration of pore former, can be used to control the release of nanosized particles through porous membranes. The membrane porosity determines the value of the membrane retardation factor, R (defined below), and is used to regulate the release rate from the pharmaceutical composition. Typical values of R for the solvent-based membranes are given in Table 1 herein.

Gradient former

The pharmaceutical composition normally also comprises at least one pharmaceutically acceptable excipient that is a gradient former, i.e. it enables a diffusion gradient to be established that results in the release of the active substance in the form of nanosized particles.

A gradient former is involved in establishment of a rate balance between the diffusion of solvent into the pharmaceutical composition and the diffusion of solute plus the outflow of the nanosuspension from the pharmaceutical composition through pores in the diffusion-controlled membrane.

Ideally, in the method according to the present invention, at least one of the pharmaceutically acceptable excipients is a water-soluble substance. Examples of suitable gradient formers are water-soluble substances like e.g. hexoses and pentoses such as, e.g. glucose, fructose, mannose, arabinose, disaccharides such as, e.g., saccharose, maltose, lactose, oligosaccharides such as, e.g., maltotriose, sugar alcohols such as, e.g., mannitol, sorbitol, xylitol, low-viscosity polymers such as, e.g., polvvinylpyrrolidone, maitodextins, dextrans, carboxylic acids such as, e.g., acetic acid, citric acid, tartaric add, fumaric acid, lactic acid and their sodium or potassium salts, sodium, potassium or calcium salts of strong acids such as, e.g. sulphuric, hydrochloric or phosphoric acid, and neutral compounds such as urea, or mixtures thereof.

One or more gradient formers may be incorporated in the composition (in the interior of the composition, i.e. in the core) and the total concentration of gradient formers in the core has to be selected in each specific formulation to obtain the desired release profile. Typically, the total concentration of gradient former present in the interior of the composition (core) is 0-95% w/w, such as e.g. 5-50% w/w, 10-70% w/w or 25-60% w/w.

At least one of the pharmaceutically acceptable excipients is included in the composition in order to ensure a formation of a nanosuspension of the active substance within the composition. In one aspect, the pharmaceutically acceptable excipient creates a suitable surface charge (Z potential) of the nanosized particles at the ionic strength and pH present in the composition when the composition is contacted with the aqueous solvent

Other pharmaceutically acceptable excipients

In the present context the term “pharmaceutically acceptable excipient” is intended to denote any material that is inert in the sense that it substantially does not have any therapeutic, prophylactic and/or diagnostic effect per se. A pharmaceutically acceptable excipient is normally used in order to obtain suitable technical properties of the final pharmaceutical composition.

A pharmaceutical composition based on the present release mechanism may further contain one or more pharmaceutically acceptable excipients. Suitable excipients are e.g. buffering agents like e.g. carboxylic adds such as, e.g., acetic acid, citric add, tartaric add, fumaric acid, lactic acid and their salts with sodium or potassium, sodium, potassium or calcium salts of strong adds such as, e.g. sulphuric, hydrochloric or phosphoric add, stabilizing agents such as, e.g., polymers such as, e.g. PVP, PEG or PEO, surface-active agents or surfactants like e.g., C3 to C20 fatty acid salts such as e.g. salts of citric acid, caprylic acid, lauric acid, palmitic acid, stearic add, oleic acid, linolic add, linoleic acid or arachidonic acid, C3 to C20 fatty acid sulphonates such as, e.g., capryl sulphonate, caprylic sulphonate, lauryl sulphonate, palmityl sulphonate, stearyl sulphonate, oleyl sulphonate, linolic sulphonate, linoleic sulphonate or arachidonic sulphonate, phosphatidyicholines, fatty acid PEO esters or ethers or other surface active agents such as, e.g., poloxamers, lecitin, suifosuccinates, anionic emulsifying waxes, non-ionic emulsifying waxes, sorbitan esters or cationic surfactants. Other pharmaceutically acceptable excipients include fillers, diluents, disintegrants, binding agents and lubricants.

Examples of suitable fillers, diluents or binders are e.g. sucrose, sorbitol, mannitol, lactose, microcrystalline cellulose, hydroxypropylcellulose, hydroxypropylmethylcellulose, dextrins, maltodextrins, starches and modified starches, sodium chloride, sodium phosphate, calcium phosphate, calcium sulphate, calcium carbonate, gelatine, polyvinylpyrrolidone and sodium carboxymethylcellulose.

Suitable disintegrants are e.g. cellulose derivatives including microcrystalline cellulose, hydroxypropylcellulose, starches such as, e.g., potato starch, croscarmellose sodium, sodium carboxymethylcellulose, alginic acid, alginates, polyvinylpyrrolidone etc.

Examples of lubricants or glidants include stearic acid, metallic stearates, waxes and glycerides, colloidal silica, sodium stearyl fumarate, polyethylene glycols and alkyl sulphates.

Other generally employed pharmaceutically acceptable excipients include wetting agents, pH adjusting agents, surface-active agents, stabilizing agents, preservatives, colouring agents and/or taste-masking agents.

Surfactants or surface active agents, suspending agents, fillers, diluents and disintegrants may be employed in order to enable a suitable nanosuspension to be formed within the interior upon intrusion of an aqueous medium.

Description of the release of nanosized particles

In the following is given a description of the release mechanisms employed by the method of the present invention. For any relevant composition, the principles can be used in order to determine the individual parameters that are important for the release (e.g. the hydrodynamic coupling factor, H; the coating retardation faction, R; the surface area of the composition, A; the membrane thickness, T; etc.). Once these parameters have been determined, it is possible by simulation to envisage how the release profile will change e.g. if the concentration of pore-forming material is increased. Examples are given herein of how to simulate release profiles based on changes in e.g. the surface area of the composition. Accordingly, the present invention provides a relatively simple model to predict the release of the active substance in the form of nanosized particles from a diffusion-coated composition and the model may also be used to design a composition that has a specific release pattern. In other words, the present invention provides an alternative method for controlling release of active substances and provides a tool in the form of a model that can be applied in designing specific compositions with predetermined release patterns.

The core contains gradient former(s) (g=g1+g2+g3) and drug molecules in the shape of nanosized particles (n). The size of the nanosized particles are described by a size distribution Pn(d) and is more easily discussed as the volume mean particle size (d). The core is coated by a porous DOV-membrane of a thickness (L). The course of events for the preparation when administered (in vivo or in vitro) can be described by three main phases:

1) Lag time: The pores in the membrane are filled with water (by dissolving the pore forming substance) and the water penetrates into the core to dissolve a substantial amount of the solid core. During this phase the volume of the pharmaceutical composition increases due to hydration of the core.

2) Steady state: The composition of the interior liquid is approximately constant due to the similar rate of dissolution of the core and release of diffusants. The volume of the pharmaceutical composition is constant in this phase.

3) Pseudo steady state: The concentration of diffusants in the core is decreasing when the core has been dissolved.

This dissolution process defines the composition of the liquid inside the membrane. This liquid, or the concentration of a certain component in this liquid, will be referred to using the subscript inside. For example: (Cg)_{inside} will mean the concentration (mg/cm³) of gradient former in the liquid inside the membrane.

Transport of matter out from a DCV-membrane-coated pharmaceutical composition occurs through diffusion. Since the membrane contains many and relatively large pores of varying size, it is permeable to all diffusants: water, gradient former and nanosized particles.

Binary diffusion (two species involved) is most common described by the Fickian approach where the rate of diffusion is described by one diffusion coefficient and the concentration gradient For multicomponent diffusion (when more than two species are involved) the situation is much more difficult and tedious to describe. (n−1)² diffusion coefficients and n concentration gradients are needed to describe the process, reference “Multicomponent diffusion”, E. L. Cussler, Elsevier Scientific publishing company, Amsterdam 1976. The mutual diffusion coefficients are in addition difficult to predict and have to be determined experimentally for each particular case. This also applies to the Stefan-Maxwell approach, reference “The Maxwell-Stefan approach to mass transfer”, Krishna-Wesselingh, Che. Eng. Sci., Vol.52, No.6, pp.861-911, 1997. Although these complex models for multicomponent diffusion have the potential to describe the situation in good agreement with experimental data, they are not usefull industrially and the present inventors have developed a simpler model which applies to the specific situation in the DCV-nano.

The model takes the binary Fickian approach modified with approximations for the special case of three diffusants, water, gradient former and nanosized particles (FIG. 4). In the approximation, the diffusion coefficients are treated as constants. The flux of the nanosized particles depends on the binary flux of the pair, gradient former and water. This flux is described by Ficks law. Note that the diffusion coefficient (D_{g}) describes the net flow of gradient former out from the core as well as the net flow of water in to the core. Depending on the nature (ingredients, concentration etc.) of the composition and phase of release, different relationships between the diffusant concentrations can be made. During steady state, using Ficks law, equation I is obtained.

$$\begin{matrix}\frac{J}{Λ} = D \cdot \frac{Δ C}{L} & ( Ficks law ) \\ {J}_{g} = \frac{A \cdot {D}_{K}}{R} \cdot \frac{{C}_{K}}{L} & ( I )\end{matrix}$$

where (J_{g}) is the net flux of the gradient former (mg/s), (A) is the membrane surface (cm²), (R) is the retardation factor of the membrane (dimensionless), (L) is the membrane thickness (cm) and (D_{g}) the diffusion coefficient of the diffusant in water (cm²/s). The concentration difference of the gradient former is approximately equal to the concentration inside the membrane (C_{g}) by assuming sink conditions (C_{g}=0) outside the membrane (mg/cm³).

The description is shown for the example with three diffusants but can be modified to more dtffusants using same approximations (i.e. the different gradient formers are coupled to the different types of nanosized particles present).

During steady state the concentration of gradient former and the density inside the membrane will be approximately constant as long as some gradient former still remains undissolved. The same approximation can be made for short times. The concentration inside the membrane in tablets can be saturated or a lower concentration that is a result of the balance between the core dissolution rate and the diffusive flux out through the DCV membrane. During the steady state, the release rate of gradient former will thus be approximately constant.

The retardation factor (R) is a factor that describes the membrane retardation towards diffusion. (R) can be different for different diffusants but is here assumed to be independent of the diffusant, which is a good approximation in most cases (R) is composed of the porosity, tourtosity, partition coefficient and the boundary layers at the membrane interfaces. The values of (R) for some different porosities (levels of pore former) for the solvent-based DCV-membrane have been determined and are given in Table 1.

TABLE 1
The retardation factor for the solvent based DCV membrane
determined using equation (I)
for different levels of membrane porosities.

| Weight % of insoluble material | |
|---|---|
| (i.e. polymer and plastiziser) in | |
| membrane composition | R-factor |
| 18.5 | 6 |
| 23.5 | 9 |
| 28.5 | 13 |
| 32.9 | 16 |
| 38.8 | 18 |

We now assume that the nanosized particles are hydrodynamically coupled and dragged along with the flux of the gradient former. The degree of hydrodynamic coupling is described by a factor (H), see below. The nanoparticle flux can be written as the sum of two contributions: the flux induced by the gradient former (Jₙ^{M.coupled}) and the binary Ficklan diffusive flux of nanosized particles (Jₙ^{Binary}). Jₙ^{H.coupled} can be seen as some kind of convective flow and is influenced by the flux of the gradient formner. This influence is described by (H), the hydrodynamic coupling factor, in equation (II). (H) is expected to be largely concentration dependent.

$${J}_{n}^{H . coupled} = H \cdot {J}_{g} \cdot \frac{{C}_{n}}{{C}_{g}} \qquad ( II )$$

The binary diffusion coefficient of the nanosized particles (only considering water and nanosized particles) can be calculated by Stokes-Einstein equation (d=nanoparticle diameter, n=the solvent viscosity, kₐ=the Boltzmann constant):

$${D}_{n} = \frac{{k}_{B} \cdot T}{3 \cdot π \cdot η \cdot d} \qquad ( Stokes \text{-} Einstein equation )$$

The flux from the “binary diffusion” of the nanosized particles can then be calculated from Ficks law and combined with equation (II) to give equation (III), which describes the combined flux of nanosized particles.

$${J}_{n} = {J}_{n}^{H . coupled} + {J}_{n}^{Binary} = H \cdot {J}_{g} \cdot \frac{{C}_{n}}{{C}_{g}} + \frac{{D}_{n} \cdot A \cdot {C}_{n}}{R \cdot L} \qquad ( III )$$

The release rate of nanosized particles (and gradient former) can be varied by changing the properties of the core (dissolution rate, solubility, amount and type of gradient former), and properties of the membrane (membrane resisitvity (R), area (A) and thickness(L)). Encapsulating the core with a membrane thus enables many possibilities to control the release from the preparation. Assuming use of both tablets and pellets a large variety in release rate can then be obtained.

It is possible to obtain approximate values of (Jₙ) at steady state (e.g. from Example 2 herein) by picking the largest value of release rate from Table 2.

TABLE 2
Release rate (%/h) of nanosized particles calculated from Example 2 at
different time intervals in the early stages of the release profile.

| | Composition | | |
|---|---|---|---|
| | A | B | C |
| Time interval | (51.9% sorbitol) | (41.9% sorbitol) | (26.5% sorbitol) |
| h | %/h | %/h | %/h |
| 0-1 | 1.7 | 3.1 | 0.3 |
| 1-2 | 35.2 | 14.5 | 1.0 |
| 2-3 | 13.9 | 22.8 | 1.5 |
| 3-4 | 10.3 | 11.9 | 0.9 |

It is also possible to calculate the expected release rate of sorbitol (gradient former in Example 2) by using equation (I). Transforming the release rate of sorbitol to the unit %/h allows an estimate of the hydrodynamic coupling factor H by simply calculating the ratio Jₙ/J_{g} according to equation (II). The values are given in Table 3 below.

It can be seen for this particular preparation that (h) varies drastically with the concentration of the gradient former. It can be expected that (H) is strongly concentration dependent in general but the dependence should be different for different preparations (i.e. that the membrane, nanoparticle, gradient former etc. influence the dependence).

TABLE 3
Expected release rate of sorbitol calculated from equation (I)
The value of H was obtained using equation (II).

| Example | A | B | C |
|---|---|---|---|
| D (cm²/s) | 1.0 × 10⁻⁵ | 1.0 × 10⁻⁵ | 1.0 × 10⁻⁵ |
| C_{sorbitol} (mg/cm³) | 628 | 489 | 292 |
| A (cm²) | 1.56 | 1.56 | 1.56 |
| R (-) | 10 | 10 | 10 |
| L (cm) | 0.015 | 0.015 | 0.015 |
| J_{sorbitol} (mg/h) | 235 | 183 | 109 |
| J_{sorbitol} (%/h) | 37 | 37 | 37 |
| Jₙobs (%/h) | 35 | 23 | 1.5 |
| H | 0.94 | 0.61 | 0.04 |

The model developed above can now be used to predict the steady state release-rate for some pharmaceutical preparations, applying realistic values to the different parameters. Typically, the value range of R is from about 2 to about ≦40, L is from about 0.002 to about 0.02 cm and A is from about 0.4 to about 40 cm². The membrane area of coated preparations can vary over a wide range, where multiple unit preparations have the largest area. Predictions of equation (III) are presented in three diagrams (FIGS. 5-7). The effect of the membrane retardation factor (R) and different combinations of C_{g}/Cₙ, H A and L on the release rate at steady state can be seen. The curves in FIG. 5 are based on values for a typical tablet while FIG. 6 and FIG. 7 display the predictions for compositions with large and small multiple units, respectively. D_{g} is put to 1×10⁻⁵ cm²/s and Dₙ to 5×10⁻⁸ cm²/s. The value of the hydrodynamic coupling parameter H is set to 1 or 0.1 in these model calculations. For the case of sorbitol as gradient former and Aquacoat as nanosized particles these values of H correspond to an approximate gradient former concentration of 600 or 300 mg/cm³, according to Table 3.

FIGS. 5-7 show that the present invention (exemplified by the model and based on experimental results) can be used to formulate a pharmaceutical preparation to get a wide range of desired release-rates at steady state. This can be achieved by varying the amount of gradient former (that influences H, the coating retardation factor (R), the formulation area (A) and membrane thickness (L). It is possible to design a composition that has a release rate in a very broad range such as from approximately 5%/h to a very fast release-rate.

In addition, the model predicts that it is possible to release nanosized particles from a pellet preparation With a porous coating Without the use of any gradient former (FIG. 7). The diffusion of the nanosized particles alone is in this case sufficient to obtain a realistic release rate of nanosized particles. Hence, the invention relates a method for designing a pharmaceutical composition coated with a diffusion membrane, said composition releasing nanosized particles comprising the active substance at a predetermined rate, the method comprising determination of a suitable retardation factor (R), a suitable hydrodynamic coupling factor (H), a suitable thickness for the diffusion membrane (L) and suitable diffusion coefficients for the ingredients in the composition and water by means of Equations I, II, III. Additionally, a method is disclosed for designing a pharmaceutical composition coated with a diffusion membrane, said composition releasing nanosized particles comprising the active substance at a predetermined rate, the method comprising simulating the release rate by varying retardation factor (R), hydrodynamic coupling factor (H), thickness of the membrane (L) and surface area of the composition (A) by means of equations I and III in order to determine which concentration of a pore-forming substance in the membrane and which concentration of a gradient former in the composition will give the predetermined rate.

### METHODS AND MATERIALS

Diffusion cells

The diffusion calls employed in the following examples were cells that simulate coated cores. A cell 5 is made of Plexiglas in which a cylindrical hole 6 (d=10 mm, h=16 mm) is made. It is possible to attach membranes 1 on each side of the hole, but in some of the following examples a membrane was only attached to one side and the other side was blocked with an inert material 4. FIG. 8 illustrates a cell employed. Such cells-simulating tablets—are used in the release experiments in the example below

The membrane is fixed by screwing a perforated plate 2 on the cell with screws 3. The cell can be filled with a solution or a suspension. The membranes used were obtained from coated tablets.

Sample preparation. One hole of the cell was blocked with either a membrane or an inert material. The diffusion cell was placed on a table with the open hole pointing up. The cell was then filled with nanosuspension. Finally, a membrane was placed to cover the open hole, the steel plate was placed above and tightened with the screws. Immediately after the preparation the diffusion cell was placed in the USP dissolution apparatus.

The membranes employed in the diffusion cell experiments are isolated from tablets that have been coated with different types of coatings, i.e. membranes. A coated tablet was put in water for a few hours. The coating was split in two with a pair of scissors. A 12 mm punch was used to isolate membranes with 12 mm diameter. The membrane was then ready to carefully be attached on the diffusion cell.

Coating of tablets can be achieved in many different ways and using various equipments. Tablets are commonly coated in a perforated pan. The pan is rotating which sets the tablets in motion. The coating composition is sprayed onto the tablets. A large flow of temperate air continuously dries the tablets during coating.

Small sized tablets and pellets are usually coated in a fluid bed. A large flow of air both sets the tablets or pellets in motion and dries them during the coating process.

Water based porous DCV membranes

A water based porous membrane was obtained by coating tablets with a coating composition containing

71% w/w of potassium hydrogen tartrate as a pore-forming substance

29% w/w of a substantially water-insoluble polymer (an Eudragit NE30D latex containing

30% w/w dry matter was employed)

based on dry matter content in the coating composition.

The particle size of the pore forming substance employed was about 10 μm. The pore-forming substance is suspended in water and mixed with the polymer latex The mixture is diluted with water to 15% w/w dry matter and applied to the tablets as a coating suspension. The pore forming agent is only sparingly soluble in the aqueous phase of the coating suspension. The concentration of pore forming substance may be varied from 0 to about 90% w/w of the dry coating. Other ingredients may be added such as, e.g., pigments, surface active agents, preservatives, plasticizers and glidants

Tablets are coated with the coating suspension. When the coated tablets are contacted with water, the pore-forming substance dissolves and accordingly, a multiplicity of pores are formed in the coating. In other words, a porous membrane is then obtained that completed surrounds the tablet (or whenever relevant the pellets).

Organic solvent based porous DCV—membranes

The coating composition may contain different water-insoluble polymers. Normally, a PVC terpolymer is employed containing 31 parts of PVC (polyvinyl chloride), 1 part of PVAc (polyvinyl acetate) and 2 parts of PVOH (polyvinyl alcohol).

The coating employed contains (as dry matter):

73.6% w/w saccharose

19.8% w/w of the PVC terpolymer described above

2.2% w/w ATBC (acetyltributylcitrate) as plasticizer

1.7% w/w Castor oil

2.7% w/w sodium bicarbonate

The particle size of the pore-forming substance employed was about 10 μm. The polymer is dissolved in acetone and ATBC and polymerised Castor oil is added. The pore-forming substance (micronised saccharose) and sodium bicarbonate are suspended in the acetone. The final concentration of dry matter in the coating dispersion is 15% w/w.

Filter membranes

The filter membrane was made of cellulose acetate with the pore size of 1.2 micrometer, thickness of approximately 0.013 cm and manufactured by Sartorius.

### EXAMPLES

The following examples further illustrate the invention and are not intended to limit the invention in any way.

### Example 1

Release of nanosized particles from diffusion cells equipped with different porous membranes

Nano suspensions were obtained using a latex from FMC Corporation (trademark Aquacoat EDC). The particle size (median particle size) was measured by laser light scattering and determined to be 130 nm both before and after the release study.

Nano suspension for the water based membrane and the filter membrane

| | |
|---|---|
| Aquacoat EDC | 5% w/w (dry matter) |
| Sorbitol | 50% w/w |
| Water | 45% w/w |

Nano suspension for the acetone based membrane

| | |
|---|---|
| Aquacoat EDC | 5.6% w/w (dry matter) |
| Sorbitol | 47.4% w/w |
| Water | 47% w/w |

1.2 ml of the nano suspensions was filled into two diffusion cells with a water-based membrane, into two cells with an acetone based membrane and into one cell with an filter membrane.

A cell was placed in a USP dissolution vessel at 37° C. Automatic analysis was performed after 24 min, 1 h, 2 h, 3 h and every hour for at least 16 h and where the amount of released nanoparticles was measured by spectrophotometry. The dissolution test was made as a normal dissolution test on tablets. The test method was not validated but performed according to standard USP dissolution test method. The dissolution medium was water at 37° C. One diffusion cell was placed in one USP dissolution vessel.

The results are shown in Table 4 and FIG. 9.

TABLE 4
Release results

| | Water | | Acetone | Acetone | Filter |
|---|---|---|---|---|---|
| Membrane | based | Water based | based | based | membrane |
| Replicate | 1 | 2 | 1 | 2 | 1 |
| Time (h) | Amount released (%) | | | | |
| 1 | 6 | 7 | 30 | 28 | 40 |
| 2 | 28 | 33 | 50 | 47 | 59 |
| 4 | 63 | 62 | 74 | 70 | 76 |
| 8 | 85 | 85 | 90 | 87 | 86 |
| 16 | 94 | 98 | 96 | 95 | 95 |

The results show that nanosized particles are released from the nanosuspension through the different membranes, Furthermore, the particle size remains constant. i.e. the particle size of the nanosized particles is the same in the nanosuspension inside the cell as outside the cell, after it has passed through the membrane. All the particulate material is released after 16 hours.

### Example 2

Release of latex nanosized particles from diffusion cells equipped with water based porous membranes—comparison of nanosized particles with different levels of gradient former.

A nanosuspension from FMC Corporation (Ethylcellulose, trademark Aquacoat EDC, solid content 30%) was used as model nanosized particles in the experiment conducted. The median particle size was measured by laser light scattering and was found to be approximately 150 nm for Aquacoat ECD prior to as well as after the study.

Three different levels of gradient former were tested, in order to investigate any effect on the release profile. Sorbitol was employed as gradient former. The sample compositions are shown below.

| | 26.5% sorbitol | 41.9% sorbitol | 51.9% sorbitol |
|---|---|---|---|
| Composition | (% w/w) | (% w/w) | (% w/w) |
| Aquacoat ECD | 29.4 | 23.3 | 19.2 |
| Sorbitol | 26.5 | 41.9 | 51.9 |
| Water | 44.1 | 34.9 | 28.8 |

Mini-diffusion cells were placed in a USP dissolution vessel with water at 37° C. and 50 rpm paddle speed. Automatic analysis was performed after 30 min, 1 h and thereafter every hour for at least 16 hours. The amount of nanosized particles released was measured by spectrophotometry at 236 nm. The test was performed according to a standard USP dissolution test method for tablets. The results are given in FIG. 10.

A significant difference in amount released substance was found for the Aquacoat samples containing 26.5% and 41.9% sorbitol, whereas the 41.9% and 51.9% samples showed similar release profiles. This indicates that the level of gradient former is a factor that influences, and thus contributes to controlling the release of nanosized particles through porous membranes.

### Example 3

Release of silica nanosized particles through porous water based membranes

A silica nano-dispersion from Eka Chemicals, Akzo Nobel (trademark Bindzil 50/80. solid content 50%) was used in another experiment to demonstrate the applicability of the concept to other types of nanosized particles. The particle size of Bindzil 50/80 was measured by laser light scattering and was found to be approximately 110 nm. The silica dispersion was mixed with a selection of excipients commonly used for tablet production. This mixture (composition given below) was then introduced into the diffusion cells. The cells were subsequently sealed with water-based membranes of the same type as used in Example 2 (above).

FIG. 11 demonstrates that silica nanosized particles can be released through porous water based membranes. It can also be seen that the dispersion with the highest level of mannitol is released faster and to a higher extent than the dispersion with low level of mannitol, which confirms the function of a gradient former and is in agreement with the theoretical model previously described. The release profile in FIG. 11 seems more extended than in FIG. 10, which could be explained by the fact that the excipient mixture used in this example can maintain a constant concentration of gradient former for a longer time than the excipient package used in Example 2. This is in agreement with mannitol being present to a level above its saturated concentration (about 17% at 37° C., Handbook of Pharmaceutical Excipients, 2^{nd} Ed., Editors. A. Wade and P. J. Weller, American Pharmaceutical Association, Washington, p 296) in contrast to the situation for sorbitol (which reaches a saturated concentration at about 66% at 25° C., Handbook of Pharmaceutical Excipients, p. 478) as used in the previous experiment,

| | Composition (Weight %) | Composition (Weight %) |
|---|---|---|
| | Low level of mannitol | High level of mannitol |
| | experiment: | experiment: |
| Bindzil 50/80 | 20 | 19 |
| Mannitol | 22 | 31 |
| Maltodextrin | 15 | 5 |
| Maltrin M150 | | |
| Kollidon, PVP25 | 2 | 2 |
| Water | 41 | 43 |

### Example 4

Release of lipid nanosized particles

Two types of solid lipid nanosized particles (SLN), manufactured by Amarin, were used as model nanosized particles in the following release experiment with water based porous membranes. The first type consisted of a lipid, Suppocire D (manufactured by Gatte-Fosse), stabillised with a negatively charged surfactant, sodium dodecyl sulphate (SDS). whereas the second consisted of Suppocire D stabillised with two neutral surfactants, Tween 40 and Span 80. Sorbitol was employed as gradient former.

Suppocire D nanosized particles were manufactured by melting the lipid together with water and surfactive agents at about 50° C. The mixture was processed in a high-pressure homogenisator at 30-90 Mpa (the pressure varied within the specified range during the process) and the temperature was kept at 50° C.

The compositions are shown below.

Suppocire D-SDS Nanosuspension for a water-based membrane

| | |
|---|---|
| Suppocire D | 5% w/w (dry matter) |
| SDS | 0.5% w/w |
| Sorbitol | 50% w/w |
| Water | 45% w/w |

Suppocire D-Tween40/Span80 nanosuspension for a water-based membrane

| | |
|---|---|
| Suppocire D | 5% w/w (dry matter) |
| Tween40 | 1.2% w/w |
| Span80 | 0.3% w/w |
| Sorbitol | 50% w/w |
| Water | 45% w/w |

In the release experiment, mini-diffusion cells with water-based membranes were placed in USP dissolution vessels, each holding 500 ml water at 37° C. and 50 rpm paddle speed. Automatic analysis was performed after 30 min, 1 h and thereafter every hour for at least 16 hours. The amount of nanosized particles released was measured by spectrophotometry at 236 nm. The test was performed according to a standard USP dissolution test method for tablets. The results are given in FIG. 12.

The results show that SLN-nanosized particles are released through the water-based membranes. The charge of the stabilising surfactant does not seem to have any significant influence on the release profile in this experiment.

### LEGENDS TO FIGURES

FIG. 1 shows the surface of cellulose acetate membrane (Satorlus) of 0.8 ,m pore size.

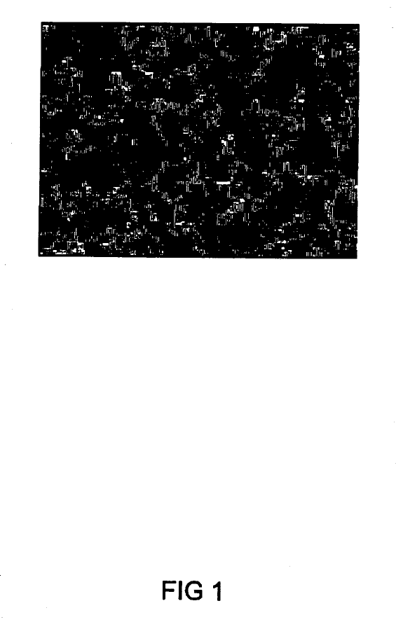

FIG. 2 shows cross-section of solvent-based membranes with a) high and b) low porosity. The solvent-based porous membranes were obtained from tablets coated with these membranes. The tablets were cut into two halves and soaked in water in order to dissolve the water-soluble substances. After another four hours, the water-soluble substances were expected to be completely dissolved and were separated from the membranes. Small fractions of membrane were cut out with razor blades, mounted onto the sample buttons and coated with a thin gold layer before the SEM-analysis was carried out.

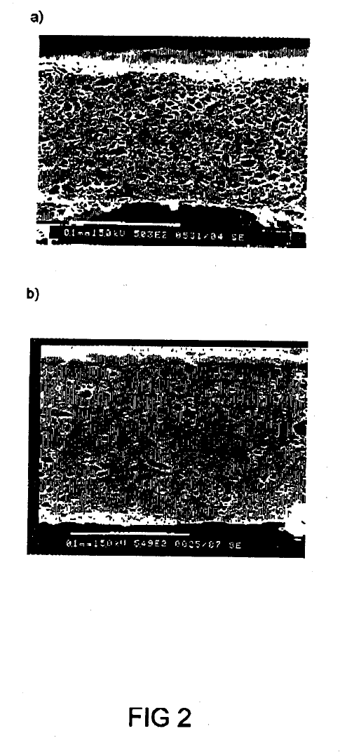

FIG. 3 shows schematically a core coated with a membrane and the diffusion of water into the core and the transport of particle out of the core

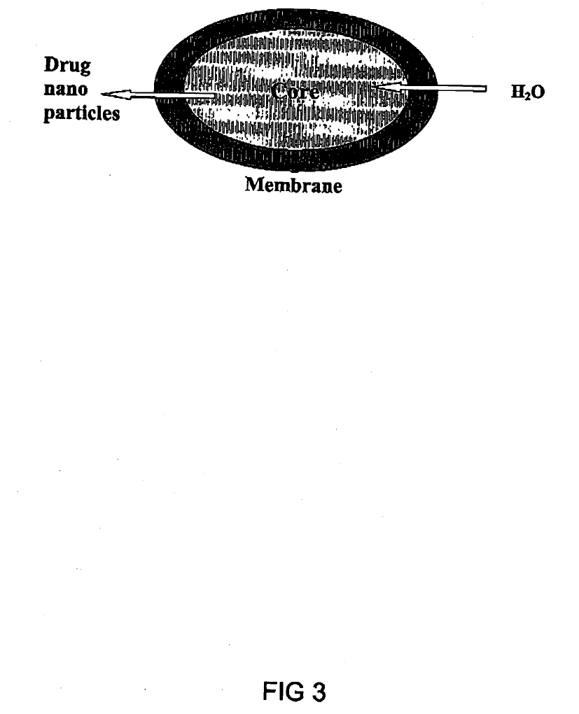

FIG. 4 shows mass transport across a DCV membrane.

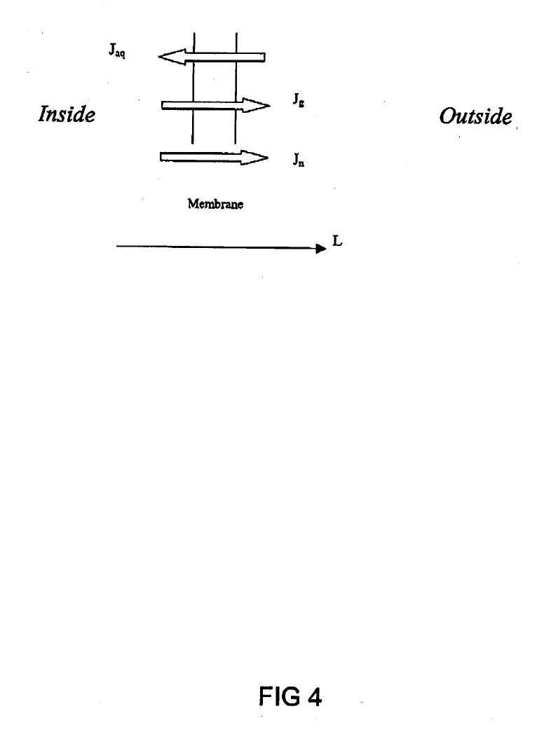

FIG. 5 shows the predicted release rate of nanosized particles at steady state for a composition containing small multiple units (A is 2 cm² and L is 0.015 cm).

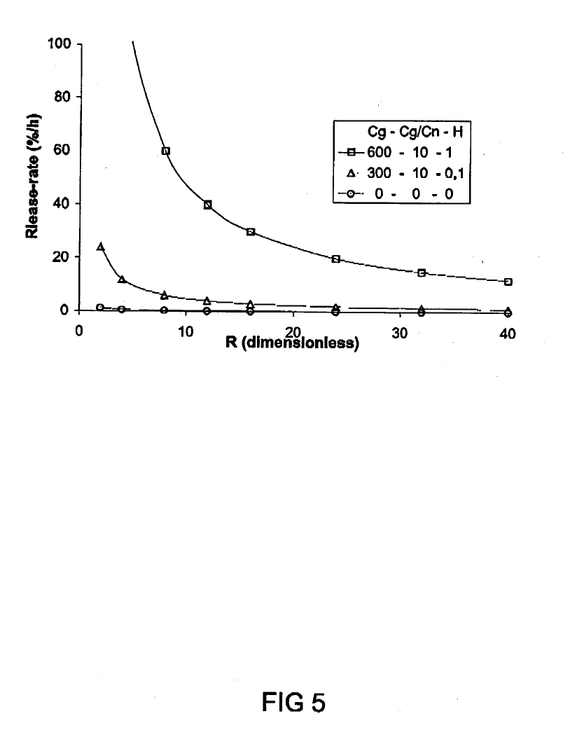

FIG. 6 shows the predicted release rate of nanosized particles at steady state for a composition containing small multiple units (A is 5 cm² and L is 0.008 cm).

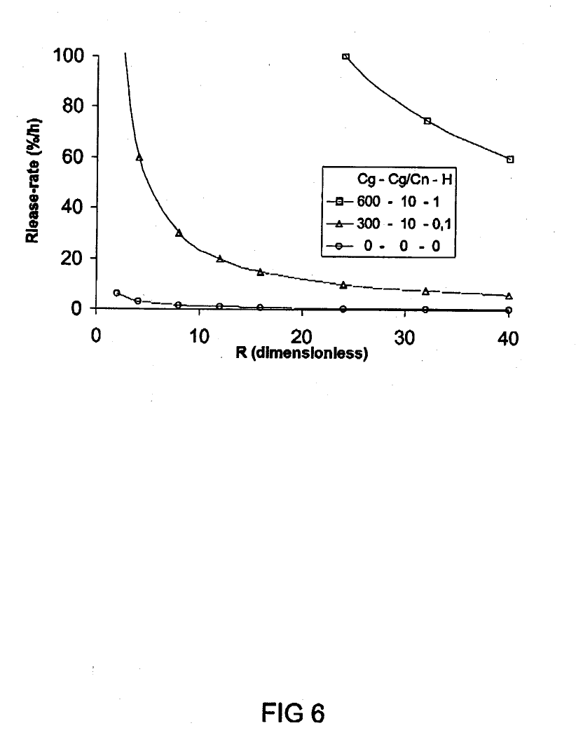

FIG. 7 shows the predicted release rate of nanosized particles at steady state for a composition containing small multiple units (A is 20 cm² and L is 0.003 cm).

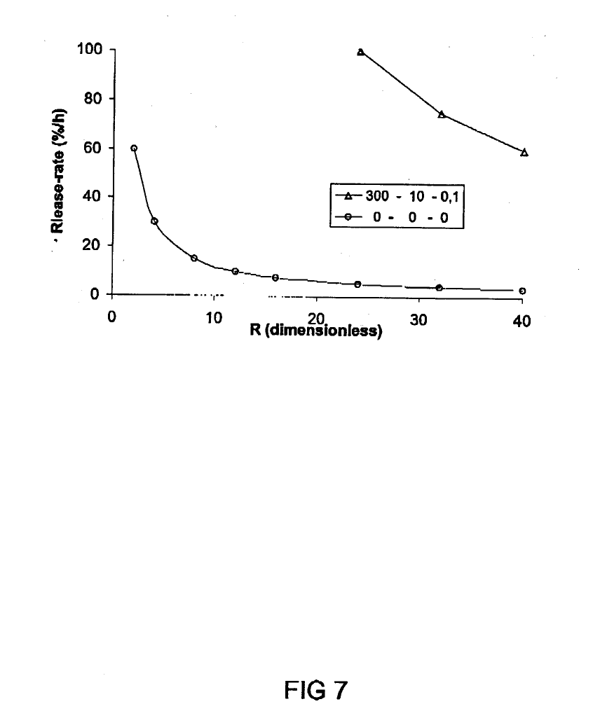

FIG. 8 shows a model of a diffusion cell that has been applied in the Examples herein. A cell 5 is made of Plexiglas in which a cylindrical hole 6 (d=10 mm, h=16 mm) is made. The membrane 1 is fixed to the cell at one end of the hole by fastening a perforated steel plate 2 on the cell via screws 3 as shown. It is possible to attach membranes 1 to each end of the cell, but in the examples a membrane was only attached to one side and the Other side was blocked with an inert material 4.

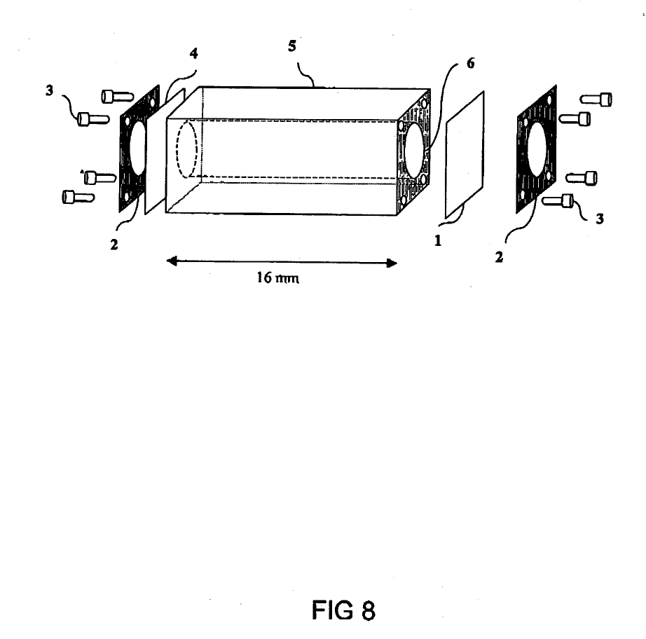

FIG. 9 shows the release rate of Aquacoat EDC through different types of porous membranes. Circles represent the water-based membrane, triangles the acetone based membrane and squares the filter membrane.

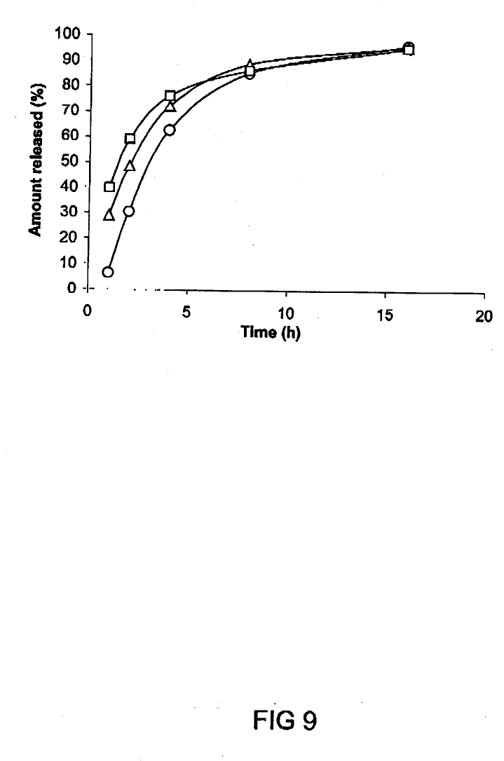

FIG. 10 shows release profile of Aquacoat-nanosized particles from diffusion cells equipped with two water-based membranes. Circles represent the 26.6%, triangles the 41.9% and squares the 51.9% sorbitol samples. The amount released has been normalised for each individual experiment with the absorbance measured after emptying the contents of the mini-diffusion cells into the dissolution bath (corresponding to the concentration at infinite time). The curves presented are the mean values of two identical experiments.

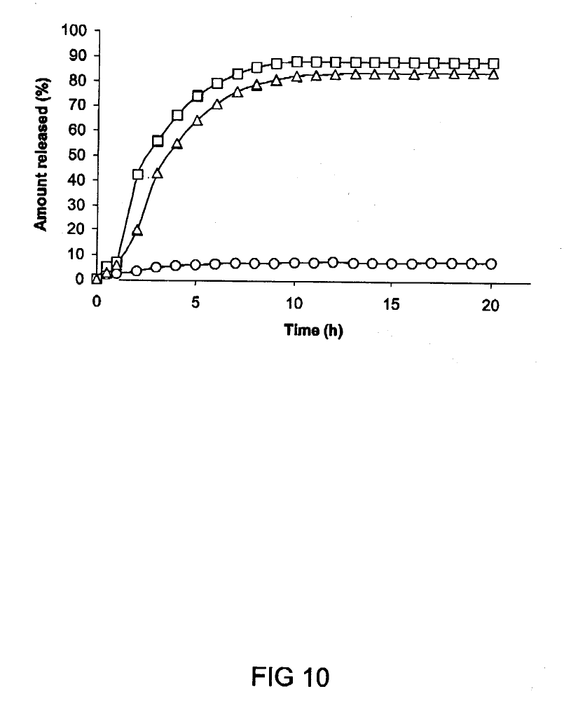

FIG. 11 shows the release profile of silica nanosized particles, Bindzil 50/80 (Eka Chemicals, Akzo Nobel), through porous water based membranes (Batch nr membranes: 02-35616 E) with different excipients. Empty squares represent the experiment with a low level of mannitol in the excipient mixture whereas the empty circles represent a high level of mannitol. The fraction released has been normalised for each individual experiment with the absorbance measured after emptying the contents of the mini-diffusion cells into the dissolution bath (corresponding to the concentration at infinite time). Each curve presented is the average value of two identical experiments.

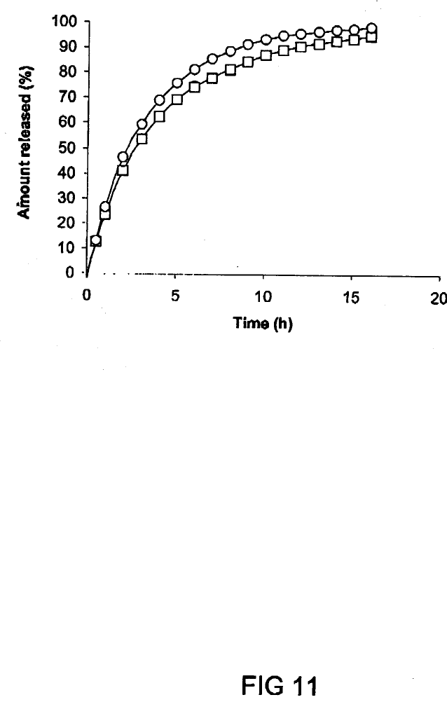

FIG. 12 shows the release profile of SLN-nanosized particles from diffusion cells equipped with water-based membranes. Squares represent the SDS particles and circles the Tween40/Span80- particles. The amount released has been normalised for each individual experiment with the absorbance measured after emptying the contents of the mini-diffusion cells into the dissolution bath (corresponding to the concentration at infinite time). The curves presented are the mean values of two identical experiments.

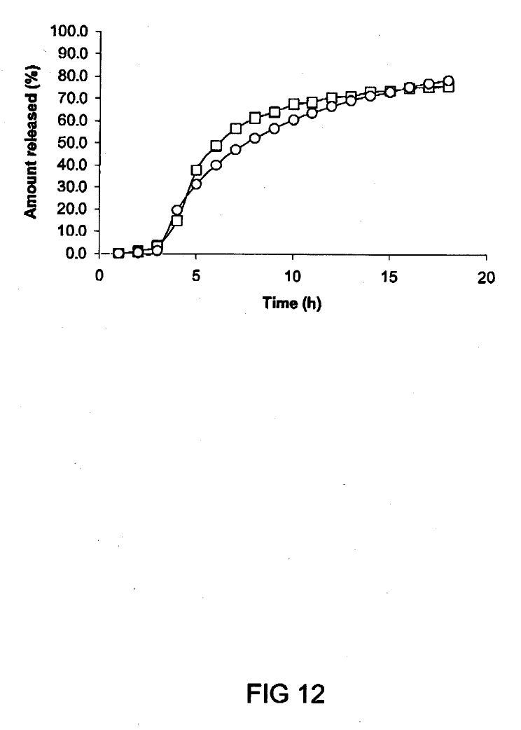

1. A method for releasing an active substance, which is substantially water-insoluble and/or immobilised on or in nanosized particles, from a pharmaceutical composition that is coated with a diffusion-controlled membrane that contains a multiplicity of pores or a pore-forming substance, the method comprising
i) contacting the pharmaceutical composition with an aqueous solvent,
ii) diffusion of the solvent into the pharmaceutical composition so that a) one or more water-soluble substances contained in the pharmaceutical composition is at least partly dissolved to obtain one or more solutes, and b) one or more substantially water-insoluble nanosized active substances or aggregates thereof, or nanosized particles containing the active substance is at least partly suspended in an aqueous medium to obtain a nanosuspension of nanosized particles,
iii) diffusion of the one or more solutes through the diffusion-controlled membrane and out of the pharmaceutical composition,
iv) establishing a diffusion gradient that enables a mass transport of the nanosuspension from the pharmaceutical composition through pores in the diffusion-controlled membrane, whereby the active substance is released from the composition.

2. A method according to claim 1, wherein the nanosized particles containing the active substance are substantially water-insoluble.

3. A method according to claim 1, wherein the nanosized particles containing the active substance are substantially water-soluble.

4. A method according to any of the preceding claims, wherein the water-insoluble active substance or the nanosized particles carrying the active substance have a water-solubility of at the most about 10 mg/ml in water at 37° C. such as, e.g., at the most about 7.5 mg/ml, at the most about 5 mg/ml, at the most about 3 mg/ml, at the most about 1 mg/ml, at the most about 0.5 mg/ml, at the most about 0.25 mg/ml, at the most about 0.1 mg/ml or at the most about 0.05 mg/ml.

5. A method according to any of the preceding claims, wherein the active substance is a therapeutically, prophylactically and/or a diagnostically active substance or a pharmaceutically acceptable salt, solvate or complex thereof.

6. A method according to any of the preceding claims, wherein the active substance is selected from the group consisting of nifedipine, felodipine, amiodipine, nisoldipine isradipine, amilodipine, nicardipine; most steroid hormones such as, e.g., estrogen, progesterone, testosterone and derivatives and analogues thereof such as desogestrel, mesterolon, ebnylestradiol, nandronlon; hormone antagonists such as tamoxifene, toremifene, flutamide, nilutamide; glucocorticoids such as, e.g., cortisone, hydrocortisone, fludrocortisone, fludocortisone, betametasone, prednisolone, budesonide, and neurological drugs such as carbamazepine. carisoprodol, prmidone, zonisamide, perphanazin, antidiabetic drugs such as glibenclamide, glimepiride, glipizide, and miscellaneous low soluble drugs such as sucralfate, padlitaxel, and acyclovir.

7. A method according to any of the preceding claims, wherein the active substance or the nano particles employed in the composition has a volume weighted median particle size of at most about 2000 nm such as, e.g., at the most about 1500 nm, at the most about 1000 nm, such as, e.g., from about 1 nm to about 1000 nm, from about 2 nm to about 750 nm, from about 5 nm to about 500 nm or from about 7.5 nm to about 500 nm, from about 10 nm to about 500 nm, from about 50 nm to about 500 nm, from about 75 nm to about 400 nm, or from about 100 nm to about 300 nm as measured by static light scattering/diffraction or dynamic light scattering.

8. A method according to any of the preceding claims, wherein the composition comprises one or more pharmaceutically acceptable excipients.

9. A method according to claim 8, wherein at least one of the pharmaceutically acceptable excipients is involved in establishment of a rate balance between the diffusion of solvent into the pharmaceutical composition and the diffusion of solute plus the outflow of the nanosuspension from the pharmaceutical composition through pores in the diffusion-controlled membrane.

10. A method according to claim 9, wherein at least one of the pharmaceutically acceptable excipients is a gradient former.

11. A method according to any of claims 8-10, wherein at least one of the pharmaceutically acceptable excipients is a water-soluble substance.

12. A method according to claim 11, wherein at least one of the pharmaceutically acceptable excipients is selected from the group consisting of hexoses and pentoses such as, e.g. glucose, fructose, mannose, arabinose, disaccharides such as, e.g., saccharose, maltose, lactose, oligosaccharides such as, e.g., maltotriose, sugar alcohols such as, e.g., mannitol, sorbitol, xyitol, low-viscosity polymers such as, e.g., polvinylpyrrolidone, maltodextrins, dextrans, carboxyic acids such as, e.g., acetic acid, citric acid, tartaric acid, fumaric acid, lactic acid and their sodium and/or potassium salts, sodium, potassium or calcium salts of strong acids such as, e.g. sulphuric. hydrochloric and phosphoric acid, and neutral compounds such as urea, and mixtures thereof.

13. A method according to any of claims 8-12, wherein at least one of the pharmaceutically acceptable excipients is included in the composition in order to ensure a formation of a nanosuspension of the active substance within the composition.

14. A method according to claim 13, wherein the pharmaceutically acceptable excipient creates a suitable surface charge (Z potential) of the nanoparticles at the ionic strength and pH present in the composition when the composition is contacted with the aqueous solvent.

15. A method according to claim 8 or 13, wherein the pharmaceutically acceptable excipient is a buffering agent like e.g. carboxylic acids such as, e.g., acetic acid, citric acid, tartaric acid, fumaric acid, lactic acid and their salts with sodium or potassium, sodium, potassium or calcium salts of strong acids such as, e.g. sulphuric, hydrochloric or phosphoric acid, stabilizing agents such as, e.g., polymers such as, e.g. PVP, PEG or PEO, surface-active agents or surfactants like e.g., C3 to C20 fatty add salts such as salts of capric acid, caprylic acid, lauric acid, palmitic acid, stearic acid, oleic acid, linolic acid, linoleic acid or arachidonic acid, C3 to C20 fatty acid sulphonates such as, e.g., capryl sulphonate, caprylic sulphonate, lauryl sulphonate, palmityl sulphonate, stearyl sulphonate, oleyl sulphonate, linolic sulphonate, linoleic sulphonate or arachidonic sulphonate, phosphatidylicolines, fatty add PEO esters or ethers or other surface active agents such as, e.g., poloxamers, lecitin, sulfosuccinates, anionic emulsifying waxes, non-ionic emulsifying waxes, sorbitan esters or cationic surfactants

16. A method according to any of the preceding claims further comprising at least one pharmaceutically acceptable excipient selected from the group consisting of fillers, diluents, disintegrants, binding agents and lubricants.

17. A method according to any of the preceding claims further comprising one or more wetting agents, pH adjusting agents, surface active agents, stabilizing agents, preservatives, colouring agents and/or taste-masking agents.

18. A method according to any of the preceding claims, wherein the pharmaceutical composition is a solid dosage form for oral use.

19. A method according to claim 18, wherein the solid dosage form is a single or a multiple-unit dosage form.

20. A method according to claim 18 or 19, wherein the dosage form is in the form of tablets, capsules or sachets.

21. A method according to any of the preceding claims, wherein the diffusion-controlled membrane comprises a substantially water-insoluble polymer selected from the group consisting of
i) cellulose derivatives including cellulose esters such as, e.g. ethylcellulose, cellulose acetate, cellulose acetate butyrate, cellulose acetate propionate, cellulose propionate, cellulose butyrate, cellulose valerate, nitrocellulose, ii) acrylic polymers such as, e.g., polymethyl methacrylate, poly(ethacrylate, methylmethacrylate, trimethylammonioethylmethacrylate chloride), poly(ethylacryiate, methylmethacrylate), iii)vinyl polymers such as e.g. polyvinyl polymers such as, e.g polyvinyl acetate, polyvinyl formal, polyvinylbutryl, vinyl chloride-vinyl acetate copolymer, ethylenevinyl acetate copolymer, vinyl chloride-propylene-vinyl acetate copolymer, polyvinyl chloride, polyvinyl chloride terpolymers, iv) other polymers such as e.g. polyethylenes, polypropylenes, polylsobutylenes, polycarbonates, polybutadienes, polyesters and other high molecular synthetic polymers and block- or copolymers and combinations thereof.

22. A method according to claim 21, wherein the diffusion-controlled membrane further comprises a plasticizer such as, e.g. acetyltributylcitrate, tributylcitrate, triacetin, acetyltriethylcitrate, triethylcitrate, oleic acid, dibutyl sebacetate, diethyl phthalate, benzyl benzoate, polyethylene glycol, triglycerides such as, e.g., hydrogenated vegetable oils, raffinated vegetable oils or glyceryl triacetate.

23. A method according to any of the preceding claims, wherein the diffusion-controlled membrane is applied on the composition in the form of a coating dispersion.

24. A method according to claim 23, wherein the coating dispersion comprises a pore-forming substance.

25. A method according to claim 23 or 24, wherein the coating dispersion comprises a dispersion of a substantially water-insoluble polymer and a water-soluble pore-forming substance and, optionally other additives like e.g. a plasticizer.

26. A method according to claim 25 wherein the coating dispersion comprises a pore-forming substance that in the coating dispersion has a solubility of at the most about 100 mg/ml such as, e.g., at the most about 50 mg/ml or at the most about 10 mg/ml at room temperature.

27. A method according to claims 24-26, wherein the pore-forming substance has a mean particle size of from about 0.1 to about 500 μm such as, e.g. from about 0.5 to about 100 μm or from about 1 to about 25 μm.

28. A method according to claim 24, wherein the pore-forming substance is selected from the group consisting of sucrose and other sugars, urea, salts such as potassium chloride, sodium chloride, calcium chloride, sodium phosphates (basic, dibasic and monobasic), potassium phosphates (basic, dibasic and monobasic), calcium sulphate, sodium sulphate, sodium citrates (basic, dibasic and monobasic), sodium tartrates (monobasic and dibasic), potassium tartrates (monobasic and dibasic), soluble polymers such as polyinyl pyrrolidone, methyl cellulose, hydroxy propyl methyl cellulose, hydroxy propyl cellulose, hydroxy ethyl cellulose, polyvinyl alcohol, chitosan, poly(butylmethacrylate), (2-dimethyl aminoethyl)-methacrylate, methyl methacrylate dextran, maltodextrin, xanthan, potassium salts, calcium salts, magnesium salts, amino acids, weak acids, carbohydrates, polymers with amino and/or acid functions and combinations thereof.

29. A method according to claim 24, wherein the pore-forming substance is selected from the group consisting of potassium bitartrate, potassium hydrogen tartrate, creatine, asparagine, glutamine, aspartic acid, glutamic acid, leucin, neroleoudne, norleucine, inosine, isoleucine, magnesium citrate, magnesium phosphate, magnesium carbonate, magnesium hydroxide, magnesium oxide, magnesium salts and combinations thereof.

30. A method according to any of the preceding claims, wherein the diffusion controlled membrane comprises potassium hydrogen tartrate as a pore-forming substance.

31. A method according to any of claims 24-30, wherein the pore-forming substance is suspended and to the major part remains undissolved in the coating dispersion.

32. A method according to any of claims 23-31, wherein the coating dispersion comprises an organic solvent.

33. A method according to any of claims 23-32, wherein the coating dispersion comprises an aqueous solvent.

34. A method according to any of the preceding claims for controlling the release of the active substance from the pharmaceutical composition.

35. A method according to claim 34 for substantially zero or first order release of the active substance during a predetermined period of time.

36. A method according to claim 34 for immediate release of the active substance.

37. A method for designing a pharmaceutical composition coated with a diffusion membrane, said composition releasing particles comprising the active substance at a predetermined rate, the method comprising determination of a suitable retardation factor (R), a suitable hydrodynamic coupling factor (H), a suitable thickness for the diffusion membrane (L) and suitable diffusion coefficients for the ingredients in tie composition and water by means of Equations I, II, III

38. A method for designing a pharmaceutical composition coated with a diffusion membrane, said composition releasing particles comprising the active substance at a predetermined rate, the method comprising simulating the release rate by varying retardation factor (R), hydrodynamic coupling factor (H), thickness of the membrane (L) and surface area of the composition (A) by means of equations I and III in order to determine which concentration of a pore-forming substance in the membrane and which concentration of a gradient former in the composition will give the predetermined rate.
